(19) United States
(12) Patent Application Publication
(10) Pub. No.: US 2007/0007149 A1
(43) Pub. Date: Jan. 11, 2007

(54) Calibration solution for conductometry

(75) Inventor: Hannes Buehler, Amden (CH)

Correspondence Address: ROTHWELL, FIGG, ERNST & MANBECK, P.C., 1425 K STREET, N.W., SUITE 800, WASHINGTON, DC, 20005, US

(73) Assignee: HAMILTON BONADUZ AG, Bonaduz (CH)

(21) Appl. No.: 11/281,895

(22) Filed: Nov. 18, 2005

(30) Foreign Application Priority Data
Nov. 18, 2004 (EP) 04 027 439.1

(51) Int. Cl. C12Q 1/00

(52) U.S. Cl. 205/777.5

(57) ABSTRACT

The present invention relates to an aqueous solution for calibrating conductometric measuring cells.

The present invention relates to a solution for calibrating conductometric measuring cells.

The measurement and monitoring of the electrolytic conductivity of solutions is an important parameter in the laboratory, in the environment and in industry Monitoring the conductivity in highly purified water, the purity of which is of very great importance in both power plants and in the pharmaceutical and semiconductor industries, is particularly important. Highly accurate electrical resistances, whose availability and accuracy do not constitute problems, are used for monitoring the measuring instruments. Calibration solutions having a precisely defined conductivity are used for testing the conductivity measuring cells. Such solutions which are to be used for the middle or higher conductivity ranges can be obtained without difficulty or can be self-prepared. However, calibration solutions for low conductivities are rarely employed since their accuracy frequently leaves a great deal to be desired. In addition, it is difficult to manipulate such solutions and their shelf-life is frequently very short.

The demands made on highly purified water are laid down, in particular, for applications in the pharmaceutical industry. Thus, by way of example, the journal “Pharmeuropa”, a medium for information from the European Pharmacopeia Commission, demands a conductivity of <5.1 μS/cm at 25° C. for aqua purificata and a conductivity of <1.3 μS/cm at 25° C. for aqua ad injectabilia.

T. Light et al. (Anal. Chem. (1993) 65, 1) describe a conductivity standard solution which contains 45-50% sucrose and acetic acid or potassium chloride.

A conductivity of <1.3 μS/cm at 25° C. is laid down for aqua ad injectabilia in the U.S. Pharmacopeia (USP) 23, 24 and 25 of the US-American Pharmacopeia.

The US “monographs” demand an accuracy of the cell constant for the conductivity measuring cells of ±2%. This provision equates with the accuracy demanded of the calibration solutions.

A variety of norms which describe measurement methodology and the preparation and/or handling of the calibration solutions exist.

The norms D 5391-93 and D 1125-95 laid down by the “American Society for Testing and Materials” (ASTM) describe different calibration solutions. The lowest conductivity is 146.9 μS/cm at 25° C. This solution consists of an aqueous 0.001M KCl solution.

The norm IEC 746, part 3, laid down by the “International Electrotechnical Commission” (IEC) likewise describes this 0.001M KCl solution of 146.9 μS/cm at 25° C.

The norm ISO 7888 laid down by the “International Organization for Standardization”, at the international level, and, respectively, the European norm EN 27888, at the regional level, also mention aqueous KCl solutions of 74 and 147 μS/cm at 25° C. No decimal place is specified. In order to prepare solutions conforming to the requirements of these norms, the water has to be boiled or freed from dissolved carbon dioxide by passing in nitrogen. There must be no contact with air when these solutions are being used.

Appropriate standard solutions having medium conductivities in the range from 147 to 12880 μS/cm, and which are accurate to between 0.5 and 1%, are offered for sale by a variety of companies.

The US metrological institute “National Institute of Standards and Technology” (NIST) markets calibration solutions having low conductivities. However, these solutions, having conductivities of 5, 15 and 26 μS/cm at 25° C., are very expensive and not always in stock. There are also a number of companies which offer standard solutions having low conductivities of 10 and 100 μS/cm at 25° C. The industry is not satisfied with these solutions, as the publication by Gingerella and Jacanin demonstrates (Cal Lab, July, August 2000, pages 29-36). Thus, this publication examined the accuracy of conductivity solutions of 10 μS/cm at 25° C. supplied by a variety of companies and found errors amounting to between 1 and 30%.

The inadequate shelf life of calibration solutions of low conductivity is understandable since they are always very dilute solutions having a salt content of less than 0.0001M. Contamination by carbon dioxide from the air can by itself cause a measurement error of approx. 1 μS/cm at 25° C. Impurities in the container or the calibration vessel and the measuring cell itself are further sources of error.

The NIST solutions of 5 and 15 μS/cm at 25° C. are based on an aqueous KCl solution to which 30% propanol has been added. As compared with corresponding solutions which are purely aqueous, the salt content can readily be increased in solutions which are only partially aqueous. Despite this, these NIST solutions have a shelf life of less than a year. In addition, their accuracy in accordance with the requirements of the USP provisions is unsatisfactory and too low.

In summary, today's calibration solutions in the low conductivity range suffer from the following limitations:

inadequate accuracy as well as a short shelf life and a high risk of contamination due to the salt concentration being too low.

The present invention is therefore based on the object of eliminating the disadvantages of the existing calibration solutions of low conductivity and of providing, in particular, stable solutions in the low conductivity range which are highly accurate, which have a long shelf life and which are at low risk of contamination. In order to achieve this object, the present invention provides a solution for calibrating conductometric measuring cells, which solution is characterized in that it comprises at least one additive which possesses at least two hydrogen bond-forming groups, with the additive being selected from the group consisting of ethylene glycol, 1,2-propylene glycol, diethylene glycol, glycerol, triethanolamine, polyethylene glycol, polypropylene glycol, polyvinyl alcohol, dihydroxy-terminated and trihydroxy-terminated polyethers and/or dihydroxy-terminated and trihydroxy-terminated polyesters. Surprisingly, it has been found that the additives which are employed in accordance with the invention make it possible to obtain calibration solutions which are highly accurate and which have a long shelf life.

The electrolytic conductivity of solutions is determined by the number and mobility of the individual ions. Calibration solutions therefore normally contain salts, e.g. KCl, which are dissociated into ions. In the case of the novel calibration solutions of the present invention, the ionic mobility of the individual ions is reduced by the incorporation of additives which cause hydrogen bonds to be formed. This makes it surprisingly possible, in contrast to the calibration solutions of the prior art which contain low salt concentrations, to have a low conductivity despite the salt concentration being sufficiently high for contamination to have little effect. Within the context of the present invention, preference is given to a calibration solution which contains additives which possess at least two hydrogen bond-forming groups and which lower the conductivity of the solution, by lowering the ionic mobility, by a factor of at least 5 as compared with solutions which do not contain additives possessing at least two hydrogen bond-forming groups.

In aqueous solutions, all ions are surrounded by a water shell. For this reason, they are described as being aquotized ions. When an ion migrates, this water shell is always entrained. For example, the small lithium ion has a large water shell and migrates more slowly than the larger potassium ion, which has a small water shell.

In the case of the calibration solutions of the present invention, the ionic mobility is preferably lowered by a part, or the whole, of the water shell being replaced with other molecules.

This is achieved, in accordance with the invention, by means of additives which form strong hydrogen bonds. Particular preference is given to using additives which possess such hydrogen bond-forming groups which enter into hydrogen bonds whose bonding energy is at least 1 kJ/mol and at most 50 kJ/mol.

Additives which possess at least two hydrogen bond-forming groups can possess groups which are selected from the group consisting of:

chloro, fluoro, hydroxyl, C₁-C₄ alkoxy, carboxyl, carbonyl, C₁-C₄ alkoxycarbonyl, amino, C₁-C₄ alkylamino, di-(C₁-C₄-alkyl)amino, cyano, carboxyamide, carboxy-(C₁-C₄-alkyl)amino, carboxy-di(C₁-C₄-alkyl)amino, sulfo, sulfido(C₁-C₄-alkyl), sulfoxido(C₁-C₄-alkyl), sulfono(C₁-C₄-alkyl), thio, nitrile, ester, nitro, disulfo, disulfido, thioether, diazoamino, triazeno, tetrazano, azido, diazo, diazirin-3-carboxy, pyridazino, hydrazino, hydrazo, aminooxy, anilino, p-toluidino, p-anisidino-, p-phenetidino, benzidino, o-tolidino, n-methylanilino, ethylenedioxy, carbonitrile, cyano, benzosulfonamido, sulfanilamido, 4-sulfamoylanilino, amidino, carboxamido, acetamido, guanidino, semicarbazido, semicarbazono, ureido, acyl, diacyl, oxamoyl, malonamoyl, succinamoyl, phthalamoyl, carbamoyl, phenylcarbamoyl, carbazoyl, allophanoyl, hydantoyl, acetoacetyl, amino acids, nucleic acids and/or proteins.

Within the context of the present invention, particular preference is given to OH groups as being hydrogen bond-forming groups since these groups are readily obtainable, inexpensive and as a rule nontoxic and interact strongly with water.

Suitable additives within the meaning of the present invention are ethylene glycol, 1,2-propylene glycol, diethylene glycol, glycerol and/or triethanolamine. Larger molecules such as polyethylene glycol, polypropylene glycol, polyvinyl alcohol, dihydroxy-terminated and trihydroxy-terminated polyethers and/or dihydroxy-terminated and trihydroxy-terminated polyesters are also suitable since they interact strongly with water.

The use of the additives triethanolamine, glycerol, polyethylene glycol and/or ethylene glycol has proved to be particularly suitable, since these additives give rise to markedly low conductivities in the range of from 1.8 to 18.9 μS/cm at 25° C. in spite of the potassium chloride concentration being, at 0.001M, 10-fold higher than in the prior art. Within the context of the present invention, particular preference is given to the additive triethanolamine since an extremely low conductivity of only 1.8 μS/cm at 25° C., and a salt concentration of 0.001M, can be achieved when using this compound. Glycerol is also particularly preferred as an additive. Particular preference is given to a calibration solution which contains, in addition to a salt, 60-95% by volume of glycerol and 5-40% by volume of water.

A range of from 0.5 to 146 μS/cm is defined as being low conductivity, while a range of from 147 to 12 880 μS/cm is defined as being medium conductivity and a range of >12 880 μS/cm is defined as being high conductivity. A particular advantage of the calibration solution according to the invention, as compared with the prior art, is that, despite its relatively high salt content of, for example, 10⁻³M, it produces a very low conductivity whereas the prior art describes calibration solutions which either exhibit low conductivities, which are then, however, at the same time accompanied by a very dilute salt content of less than 0.0001M, or exhibit a conductivity of ≧146.9 μS/cm at a salt content of 0.001M.

Preference is given, according to the invention, to a calibration solution which has a conductivity of <150 μS/cm, particularly preferably of <80 μS/cm, in particular preferably of <20 μS/cm, even more preferably of <10 μS/cm, and most preferably of <2 μS/cm.

The content of additives is preferably at least 10% by volume and at most 99% by volume, particularly preferably at least 50% by volume and at most 95% by volume, in particular preferably at least 80 and at most 90% by volume, and most preferably about 90% by volume, in each case based on the calibration solution, since, according to the invention, it was possible to detect a particularly low conductivity at this content. Within the context of the present invention, particular preference is given to a calibration solution which comprises water as solvent, that is to say an aqueous calibration solution. In this connection, the content of water preferably represents a concentration of at least 5% by volume and at most 95% by volume, particularly preferably a concentration of at least 7% by volume and at most 50% by volume, in particular preferably a concentration of at least 10% by volume and at most 20% by volume, and most preferably a concentration of about 10% by volume, in each case based on the calibration solution. In one embodiment, preference is given to water being the only solvent which is present.

In another embodiment of the invention, the calibration solution comprises water and at least one further solvent as solvents, with the solvent being selected from the group consisting of alcohol, ketone, ester, amide and/or nitrogen compounds.

The content of the at least one further solvent is preferably at least 10% by volume and at most 65% by volume, particularly preferably at least 20% by volume and at most 40% by volume, in particular preferably 30% by volume, in each case based on the solvent mixture comprising water and at least one further solvent.

According to the invention, suitable alcohols are methanol, ethanol, propanol, butanol, octanol and/or cyclohexanol; suitable ketones are acetone, butanone and/or cyclohexanone; suitable esters are acetic acid esters and/or glycol esters; a suitable amide is dimethylformamide; suitable nitrogen compounds are pyridine, N-methylpyrrolidine, nitrobenzene and/or acetonitrile. Within the context of the present invention, particular preference is given to using alcohol as solvent since this compound is readily obtainable, inexpensive and as a rule nontoxic. The advantage of using solutions which comprise at least one further solvent in addition to water is that the salt content can be further increased as compared with that in purely aqueous solutions.

A preferred embodiment of the present invention provides for the calibration solution to comprise at least one salt. This salt is required for transporting the electric current in the solution after the salt has dissociated into its ions. As a result of the addition of the salt, the solutions are also less susceptible to contamination and consequently have a longer shelf life. Thus, it was found that it was possible to leave open bottles which were filled with a calibration solution according to the invention for one hour without the conductivity of the solution being significantly altered by the entry of CO₂. The salt content therefore also has an influence on the stability of the conductivity of a calibration solution. Within the context of the present invention, it was also found, with regard to the long-term stability of a 5 μS/cm calibration solution which was stored in a 250 ml glass bottle, that the conductivity of this solution only changed by 0.03 μS/cm at 25° C., that is only very slightly, over a period of 27 months. This represents a major advantage as compared with the calibration solutions of the prior art which usually have a shelf life of less than a year.

Within the context of the present invention, particular preference is given to salts which comprise, as cations, alkali metal ions and/or alkaline earth metal ions such as lithium, sodium, potassium, rubidium and/or cesium ions and/or magnesium, calcium, strontium, barium and/or beryllium ions, and, in addition, NH₄⁺. Preferred anions are chlorides, carbonates, bicarbonates, phosphates, etc. The salt potassium chloride is particularly preferred. Particular preference is also given to the salt employed being lithium acetate.

The content of salt is also preferably at least 0.0005M (mol/l), particularly preferably at least 0.0008M (mol/l) and, in particular preferably, at least 0.001M (mol/l).

In one embodiment of the present invention, the conductivity at 25° C. of the calibration solution in the case of the abovementioned salt concentrations is preferably <150 μS/cm, particularly preferably <80 μS/cm, in particular preferably <20 μS/cm, even more preferably <10 μS/cm and most preferably <2 μS/cm.

Particular preference is given to a solution which has a content of salt, e.g. KCl, of ≧0.0005M and whose conductivity is at the same time <70 μS/cm, in particular <50 μS/cm.

In addition, the present invention provides for a method for preparing a solution for calibrating conductometric measuring cells, wherein the method comprises the following steps:
(a) providing an aqueous calibration solution,
(b) adding at least one additive to the aqueous calibration solution from (a), which additive possesses at least two hydrogen bond-forming groups, as described above.

The present invention furthermore provides for the use of a solution, which comprises at least one additive which possesses at least two hydrogen bond-forming groups, for calibrating conductometric measuring cells.

The present invention furthermore provides for the use of at least one additive, which possesses at least two hydrogen bond-forming groups, for preparing a solution for calibrating conductometric measuring cells.

The following examples illustrate the claimed advantages of the calibration solution according to the invention for conductometry.

### EXAMPLE 1

An aqueous 0.001M solution of KCl having a conductivity of 147 μS/cm at 25° C., as is described in a number of norms, serves as the basis.

| 0.001M KCl in 10% water and 90% ethylene glycol: | |
|---|---|
| Conductivity: | 18.9 μS/cm at 25° C. |
| 0.001M KCl in 20% water and 80% glycerol: | |
| Conductivity: | 3.4 μS/cm at 25° C. |
| 0.001M KCl in 20% water and 80% polyethylene glycol: | |
| Conductivity: | 16.3 μS/cm at 25° C. |
| 0.001M KCl in 10% water and 90% triethanolamine: | |
| Conductivity: | 1.8 μS/cm at 25° C. |
| 0.0013M KCl in 10% water and 90% glycerol | |
| Conductivity: | 1.3 μS/cm at 25° C. |

The exemplary embodiments demonstrate that the addition of suitable substances significantly reduces ion mobility. This thereby makes it possible to prepare calibration solutions which have very low conductivities but which nevertheless have relatively high salt contents of, for example, 10⁻³M.

### EXAMPLE 2

Since the salt content is relatively high, the influence of contaminants is correspondingly less. 250 ml bottles which are filled with these calibration solutions can be left open for 1 hour without the conductivity being altered significantly by the entry of CO₂.

### EXAMPLE 3

The following example demonstrates the long-term stability of a 5 μS/cm calibration solution which was stored in a 250 ml glass bottle. The calibration solution contained 80% glycerol, 20% water and 0.0014M KCl. The conductivity at 25° C. was determined periodically.

| Date | Conductivity |
|---|---|
| September 2000 | 4.97 μS/cm at 25° C. |
| April 2001 | 4.98 μS/cm at 25° C. |
| December 2001 | 4.99 μS/cm at 25° C. |
| June 2002 | 4.99 μS/cm at 25° C. |
| December 2002 | 5.00 μS/cm at 25° C. |

Some of these values were confirmed by the Physikalisch-Technische Bundesanstalt [German National Metrology Institute] in Brunswick.

1. A solution for calibrating conductometric measuring cells, wherein
it comprises at least one additive which possesses at least two hydrogen bond-forming groups which are selected from the group consisting of ethylene glycol, 1,2-propylene glycol, diethylene glycol, glycerol, triethanolamine, polyethylene glycol, polypropylene glycol, polyvinyl alcohol, dihydroxy-terminated and trihydroxy-terminated polyethers and/or dihydroxy-terminated and trihydroxy-terminated polyesters.

2. The solution as claimed in claim 1,
wherein
the hydrogen bond-forming groups enter into hydrogen bonds whose bonding energy is at least 1 kJ/mol and at most 50 kJ/mol.

3. The solution as claimed in claim 1, wherein
the hydrogen bond-forming groups are OH groups.

4. The solution as claimed in claim 1, wherein
the additive is triethanolamine, glycerol, polyethylene glycol and/or ethylene glycol.

5. The solution as claimed in claim 4,
wherein
the additive is triethanolamine and/or glycerol.

6. The solution as claimed in claim 1, wherein
it exhibits a conductivity of <150 μS/cm.

7. The solution as claimed in claim 1, wherein
the content of additives is at least 10% by volume and at most 99% by volume, based on the calibration solution.

8. The solution as claimed in claim 1, wherein
it comprises water as solvent.

9. The solution as claimed in claim 1, wherein
it comprises water and at least one further solvent as solvents.

10. The solution as claimed in claim 9,
wherein
the at least one further solvent is selected from the group consisting of alcohol, ketone, ester, amide and/or nitrogen compounds.

11. The solution as claimed in claim 1, wherein
the solution comprises at least one salt.

12. The solution as claimed in claim 11,
wherein
the content of salt is at least 0.0005M.

13. The solution as claimed in claim 11, wherein
the content of salt is at least 0.0005M and, at the same time, the conductivity of the solution is <70 μS/cm.

14. The use of a solution which comprises at least one additive which possesses at least two hydrogen bond-forming groups and which is selected from the group consisting of ethylene glycol, 1,2-propylene glycol, diethylene glycol, glycerol, triethanolamine, polyethylene glycol, polypropylene glycol, polyvinyl alcohol, dihydroxy-terminated and trihydroxy-terminated polyethers and/or dihydroxy-terminated and trihydroxy-terminated polyesters for calibrating conductometric measuring cells.
